(19) United States
(12) Patent Application Publication
(10) Pub. No.: US 2005/0152858 A1
(43) Pub. Date: Jul. 14, 2005

(54) Solubilizing agents for active or functional organic compounds

(75) Inventors: Steven H. Bertz, Morristown, NJ (US); Ilya Makarovsky, Fair Lawn, NJ (US); Donna N. Laura, Nutley, NJ (US)

Correspondence Address: INTERNATIONAL SPECIALTY PRODUCTS;Attn: William J. Davis, Esq., Legal Department, 1361 Alps Road, Building No. 8, Wayne, NJ, 07470, US

(73) Assignee: ISP INVESTMENTS INC.

(21) Appl. No.: 11/007,744

(22) Filed: Dec. 8, 2004

Related U.S. Application Data
(63) Continuation-in-part of application No. 10/617,497, filed on Jul. 11, 2003.
(63) Continuation-in-part of application No. 10/859,533, filed on Jun. 2, 2004.
(63) Continuation-in-part of application No. 10/952,948, filed on Sep. 29, 2004.
(63) Continuation-in-part of application No. 10/952,949, filed on Sep. 29, 2004.
(63) Continuation-in-part of application No. 10/961,564, filed on Oct. 8, 2004.

(51) Int. Cl. A61K 7/42

(52) U.S. Cl. 424/59; 514/532; 514/538

(57) ABSTRACT

An active or functional organic compound is solubilized in a diaryl organic compound having a polar or polarizable functional group therein, as a solvent, cosolvent or additive, to form a composition thereof. Representative active or functional organic compounds include those present in personal care products, e.g., sunscreens containing UVA/UVB absorbing compounds, such as avobenzone, benzophenone-3, and 4-methylbenzylidene camphor. Such compositions also show increased SPF, UVA/UVB absorbance ratio, and critical wavelength performance properties.

### CROSS-REFERENCE TO RELATED U.S. APPLICATIONS

This application is a continuation-in-part of co-pending U.S. patent applications Ser. No. 10/617,497, filed Jul. 11, 2003; Ser. No. 10/859,533, filed Jun. 2, 2004; Ser. Nos. 10/952,948 and 10/952,949 both filed Sep. 29, 2004; and Ser. No. 10/961,564, filed Oct. 8, 2004, the entire contents of which are incorporated by reference herein.

### BACKGROUND OF THE INVENTION

1. Field of the Invention

This invention relates to compositions containing an active or functional organic compound which requires solubilization, and more particularly, to such compositions which are effectively solubilized by addition of a diaryl organic compound containing a polar or polarizable functional group as solvent, cosolvent or additive.

2. Description of the Prior Art

Many commercial products, e.g., personal care (e.g., sunscreens or UV-filters), pharmaceutical, agricultural and industrial compositions, contain active or functional materials which require solubilization in the form of a solution, emulsion or dispersion, in aqueous or non-aqueous form. For example, a sunscreen formulation containing aromatic compounds such as avobenzone (Escalol® 517) and/or benzophenone-3 (Escalol® 567) as active UVA/UVB absorbing ingredients, requires a solubilization agent to keep them in an emulsion, i.e., to prevent crystallization. Several such solubilizers are known, e.g., ethyl benzoate or a C₁₂-C₁₅ alkyl benzoate; however, the former compound is a strong irritant, and the latter is only a mediocre solvent for avobenzone and benzophenone-3.

Furthermore, previous syntheses of diaryl organic esters, e.g., 2-phenylethyl benzoate, have employed toxic solvents or explosive or expensive reagents. For example, 2-phenylethanol and benzoic acid have been condensed in acetonitrile solvent with the aid of a stoichiometric N,N,N′,N′-tetramethylchloroformamidinium chloride reagent, prepared in situ from N,N,N′,N′-tetramethylurea, oxalyl chloride and pyridine (Fujisawa et al., Chem. Lett. 1982,1891-1894). (Oxalyl chloride is a toxic liquid and produces carbon monoxide, a toxic gas; pyridine has a sickening odor and adverse health effects.) Similarly, 2-phenylethanol and benzoic acid have been condensed in tetrahydrofuran solvent with the aid of a stoichiometric 3-methylbenzothiazole-2-selone/diethyl azodicarboxylate/N,N-dimethylaniline reagent (Mitsunobu et al., Chem. Lett. 1984, 855-858) or a stoichiometric triphenylphosphine/S-benzyl-S-phenyl-N-p-tosylsulfilimine reagent (Aida et al., Chem. Lett. 1975, 29-32). (The selenium and phosphorous by-products create a toxic waste problem, and tetrahydrofuran is not acceptable in personal care applications.) They have also been condensed in toluene with catalytic (ca. 7.3 mol %) toluenesulfonic acid, prepared in situ from toluene and sulfuric acid (Zardecki et al., Polish Patent, PL 55230, issued May 15, 1968). (Our strong acid procedure features a low concentration, 0.47 mol %, of methanesulfonic acid, which has a low molecular weight and produces a smaller waste stream.)

2-Phenylethyl benzoate has also been prepared from 2-phenylethanol and benzoic anhydride with alkali or alkali earth metal perchlorates (Chakraborti et al., Tetrahedron 2003, 7661-7668) as catalysts, in dichloromethane solvent with vanadium salts as catalysts (Chen, U.S. Pat. No. 6,541,659, issued Apr. 1, 2003) or with bismuth tris(trifluoromethanesulfonate) catalyst (Orita et al., Angew. Chem. Int. Ed. 2000, 2877-2879). (Perchlorates are an explosion hazard, and dichloromethane is not acceptable in personal care applications.) It has also been prepared from 2-phenylethanol and benzoic anhydride in N,N-dimethylformamide solvent with equimolar 1,1,3,3-tetramethylguanidine (Kim et al., Bull. Korean Chem. Soc. 1984, 205-206). (N,N-Dimethylformamide is not acceptable in personal care applications.)

Finally, 2-phenylethyl benzoate has been prepared from 2-phenylethanol and benzoyl chloride in acetonitrile solvent with ZnCl₂ reagent (Kim et al., Synth. Commun. 1986, 659-666) or neat with pyridine base (Tommila, Ann. Acad. Sci. Fenn., Ser. A, 1942, vol. 59, 2-34). (Zn has waste disposal problems, and acetonitrile and pyridine are toxic.)

Accordingly, it is an object of this invention to provide a composition including an active or functional organic compound, which is solubilized by a safe and effective organic compound as solvent, cosolvent or additive.

Another object is to provide a personal care, e.g., a sunscreen, cosmetic, pharmaceutical, agricultural or industrial composition containing a solid active or functional organic compound which is solubilized therein.

A further object herein is to solubilize at least 10%, preferably 20%, most preferably 30% (w/w) or more of the active with the solubilizer of the invention.

A specific object of the invention is to provide a sunscreen composition containing active UVA and/or UVB compounds, which are solubilized by an effective organic solvent.

Still another object of the invention is to provide a process for synthesis of the solubilizer compound that economically affords a product with low color and low odor and that has a low environmental impact and no dangerous (e.g., toxic or explosive) reagents or by-products.

These and other objects and features of the invention will be made apparent from the following description.

### SUMMARY OF THE INVENTION

What is described herein is a composition of an active or functional organic compound which is solubilized in a diaryl organic compound containing a polar or polarizable functional group, e.g., a phenylethyl ester which is an aryl carboxylic ester of 2-phenylethanol.

A general formula for the solubilizer compounds of the invention is shown below: 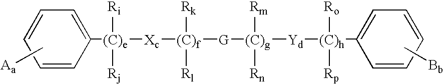
where
G=polar or polarizable functional group (e.g., ester, amide, carbonate, carbamate, urea, carbinyl, oxa, oxo, alkylidene, silyl, sulfonyl, sulfoxyl, phosphonyl, phosphinyl, etc., or thio derivatives thereof).
X_{c}, Y_{d}=G or heteroatom and any attached groups (e.g., O S, or NR_{q}, etc.).
Aₐ, B_{b}=H, F, alkyl or fluoralkyl groups, CN, CO₂Rᵣ, or heterogroups (e.g., OH, ORₛ, O₂CRₜ, NRᵤRᵥ, NO₂, F, Cl, SiR_{w}RₓR_{y}, SO₃R_{z}, etc.).
Rᵢ—R_{z}=H, F, alkyl or fluoralkyl groups (e.g., methyl, ethyl, n-propyl, i-propyl, n-butyl, s-butyl, i-butyl, t-butyl, etc., or their fluoro analogs) or alkoxy groups OR′ (R′=Rᵢ—R_{z}).
a, b=1-5
c, d=0-2
e-z=0-4.

A preferred class of compounds are diaryl esters, i.e., an aryl carboxylic acid ester of an aryl alcohol: 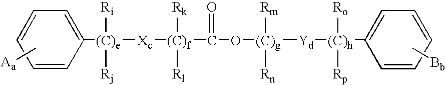
where A, B, X, Y, and R are defined as above.

Suitable compounds include aryl benzoates, having the formula: 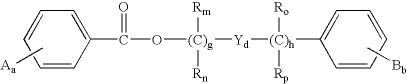
where A, B, Y, and R are defined as above.

Preferred compounds have the formula: 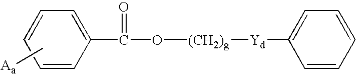
where A and Y are as defined above, and g=1-3.

In preferred forms of the invention, the ester is a 2-phenylethyl, benzyl or substituted benzyl benzoate, the active or functional organic compound is a solid organic compound, e.g., a personal care, cosmetic, sunscreen (UV filter), pharmaceutical, agricultural or industrial compound; most preferably an active sunscreen ingredient, e.g., a sunscreen composition containing UVA and/or UVB chemical compounds, e.g., avobenzone and/or benzophenone-3. Typically, the sunscreen composition exhibits increased SPF, UVA/UVB absorbance ratio and critical wavelength.

The active is solubilized in an amount of at least 10%, preferably 20%, most preferably 30% w/w or more with the solubilizer of the invention.

Another feature of the invention is the provision of processes for producing the ester derivatives, as detailed below.

### DETAILED DESCRIPTION OF THE INVENTION

The most general formula for the compounds of the invention is the following: 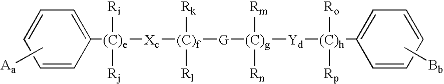
where
G=polar or polarizable functional group (e.g., ester, amide, carbonate, carbamate, urea, carbinyl, oxa, oxo, alkylidene, silyl, sulfonyl, sulfoxyl, phosphonyl, phosphinyl, etc., or thio derivatives thereof).
X_{c}, Y_{d}=G or heteroatom and any attached groups (e.g., O, S, or NR_{q}, etc.).
Aₐ, B_{b}=H, F, alkyl or fluoralkyl groups, CN, CO₂Rᵣ, or heterogroups (e.g., OH, ORₛ, O₂CRₜ, NRᵤRᵥ, NO₂, F, Cl, SiR_{w}RₓR_{y}, SO₃R_{z}, etc.).
Rᵢ—R_{z}=H, F, alkyl or fluoralkyl groups (e.g., methyl, ethyl, n-propyl, i-propyl, n-butyl, s-butyl, i-butyl, t-butyl, etc., or their fluoro analogs) or alkoxy groups OR′ (R′═Rᵢ—R_{z}).
a, b=1-5
c, d=0-2
e-z=04.

A preferred class of compounds are diaryl esters, i.e., an aryl carboxylic acid ester of an aryl alcohol: 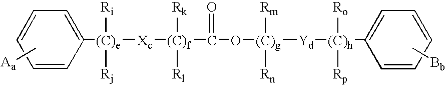
where A, B, X, Y, and R are defined as above.

Suitable compounds include aryl benzoates, having the formula: 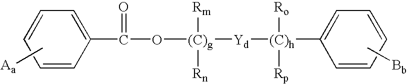
where A, B, Y, and R are defined as above.

Preferred compounds have the formula: 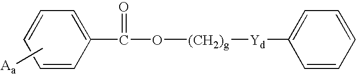
where A and Y are as defined above, and g=1-3.

Representative compounds of the invention have the named formulas shown in Chart 1: 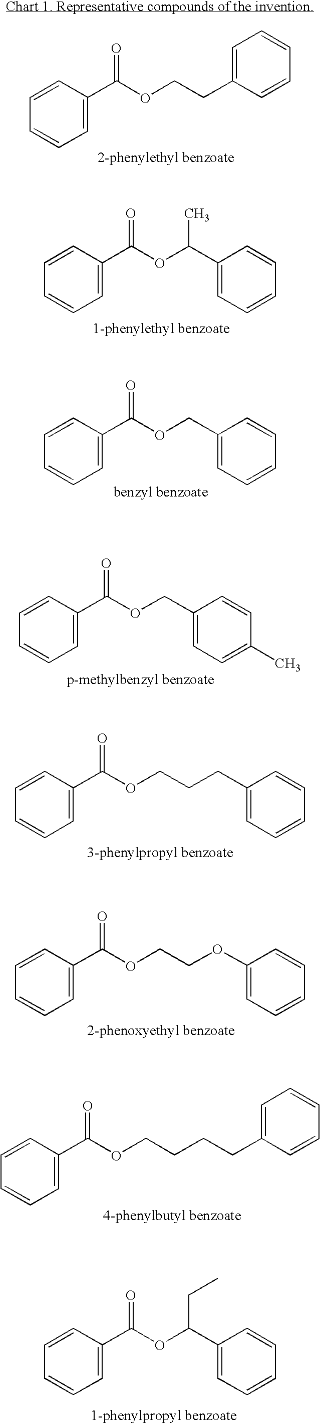 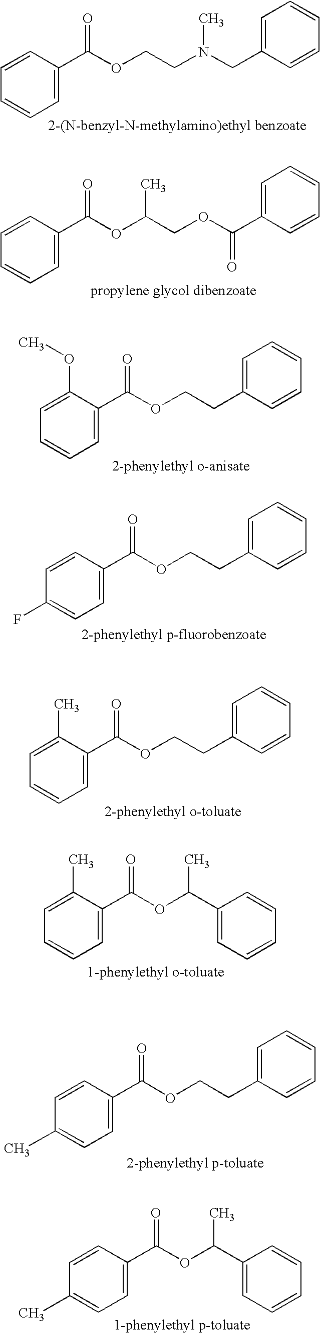 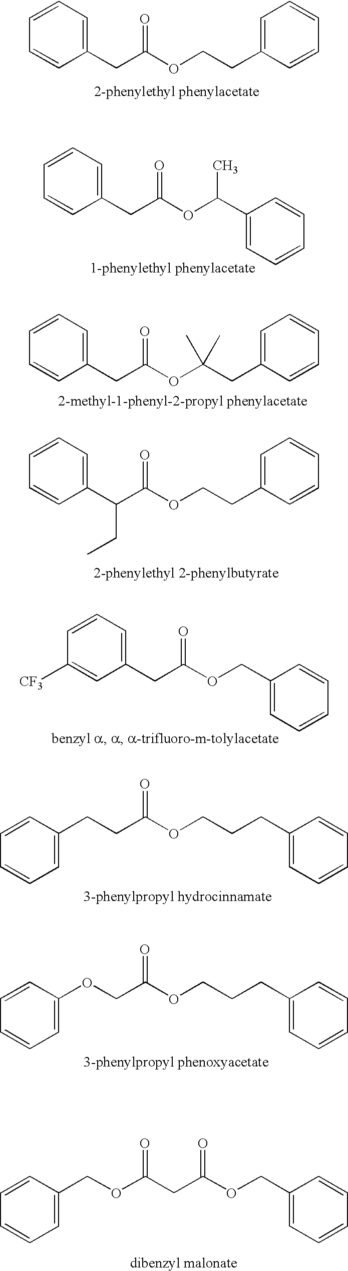
Process for Making the Solubilizer of the Invention

The process for making a typical solubilizer of the invention will be illustrated by the examples below. Accordingly, 2-phenylethyl benzoate solubilizer was prepared by reacting 2-phenylethanol (phenethyl alcohol) and benzoic acid in the presence of a catalyst, e.g., a Lewis acid catalyst such as tin oxalate (FASCAT 2001®), at temperatures above ca. 180° C., preferably at ca. 190-220° C., or a Brønsted (‘strong’) acid catalyst such as methanesulfonic acid, preferably at ca. 150-170° C. Additives such as triisodecylphosphite (TDP) and hypophosphorous acid (HPA) can improve the color of the product. Purification involves distillation of excess 2-phenylethanol or extraction of excess benzoic acid with aqueous sodium carbonate and treatment with activated carbon. Alternately, most of the products can be purified by distillation under high vacuum.

Acid chlorides, anhydrides and esters are also useful starting materials. Representative compounds of the invention are summarized in Chart 1, and their preparations are described in the Examples below.

Invention Compositions

Formulations such as sunscreen compositions containing active UVA and UVB compounds, e.g., avobenzone, benzophenone-3, and 4-methylbenzylidene camphor were effectively solubilized in 2-phenylethyl benzoate or the other compounds of the invention. Enhancement of the UVA component of their absorption spectrum relative to the UVB portion, boosting of the SPF, and increased critical wavelength were typically observed.

Other UV filter actives that may be employed in the present inventive compositions (and solubilized in 2-phenylethyl benzoate, 2-phenethyl p-toluate, benzyl benzoate, etc.) include p-Aminobenzoic acid (PABA), Camphor benzalkonium methosulfate, Homosalate, Phenylbenzimidazole sulfonic acid, Terephthalidene dicamphor sulfonic acid, Benzylidene camphor sulfonic acid, Octocrylene, Polyacrylamidomethyl benzylidene camphor, Ethylhexyl methoxycinnamate, PEG-25 PABA, Isoamyl p-methoxycinnamate, Ethylhexyl triazone, Drometrizole trisiloxane, Diethylhexyl butamido triazone, 3-Benzylidene camphor, Ethylhexyl salicylate, Ethylhexyl dimethyl PABA, Benzophenone-4, Benzophenone-5, Methylene bis-benztriazolyl tetramethylbutylphenol, Disodium phenyl dibenzimidazole tetrasulfonate, Bis-ethylhexyloxyphenol methoxyphenol triazine, and Polysilicone-15. Such compositions may include one or more of the aforementioned UV filter actives, including avobenzone, benzophenone-3, and 4-methylbenzylidene camphor (MBC).

Other actives such as personal care, cosmetic, pharmaceutical, agricultural and industrial compounds are effectively solubilized by the compounds of the invention, including such actives as antibacterial and herbicidal, e.g., algaecidal, compounds, particularly to keep the active in emulsion form without crystallizing or precipitating out of the emulsion, and without requiring the use of large amounts of solvent. Examples of such pharmaceutical compositions include one or more of Furosemide, Lovastatin, Clarithromycin, Diclofenac, Famotidine, Carbamaxepine, Dipridamole, Chlorthiazide, Spironolactone, Dilantin, Imipranine, Melfloquine, Cyclosporine, Glyburide, and Nimodipine. Compositions of the present invention may also include combinations of actives or functional organic compounds, such as, for example, a pharmaceutical (one or more thereof) and a UV filter active (one or more thereof, as well).

The invention will now be illustrated more particularly by the examples which follow:

### EXAMPLE 1

### Preparation of 2-Phenylethyl Benzoate (Lewis Acid Catalyst)

A 2-L, 4-neck, round-bottom flask, fitted with a thermometer, mechanical stirrer, nitrogen inlet tube and Liebig condenser/receiving flask, was charged with 671.7 g (5.50 mol, 1.00 equiv) of benzoic acid, 739.1 g (6.05 mol, 1.10 equiv) of 2-phenylethanol, and 2.5 g (0.2% w/w) of Fascat 2001®. The system was heated gently with slow stirring (<50 rpm) until all the benzoic acid was in solution. The air was removed with three cycles of evacuation/nitrogen fill using a mechanical vacuum pump (50-100 torr). The rate of stirring was increased to ca. 200 rpm, the nitrogen sparge was set at 0.2 scfh, and the reaction mixture was heated to 180° C. After a 1-h hold, 38.3 g of distillate had been collected. The alcohol (9.1 g) was separated and returned to the reaction mixture. The temperature was increased to 190° C. and held for 1 h; an additional 45.2 g of distillate was collected. The alcohol (16.0 g) was separated and returned. The temperature was increased to 200° C. and held for 1 h; an additional 33.5 g of distillate was collected. The alcohol (8.2 g) was separated and returned. Finally, the temperature was increased to 210° C., and the nitrogen sparge was increased to 0.5 scfh. After a 1-h hold, 21.2 g of distillate had been collected; 8.0 g of alcohol was separated, but not returned. The reaction mixture was cooled to room temperature and sampled for analysis. The acid number was 4.04 mg KOH/g (98.3% conversion), and the APHA color was 29. The excess 2-phenylethanol (4.4% by GLC) was removed by vacuum distillation at 175-180° C. (20 torr, 0.5 scfh nitrogen sweep) for 2 h. The APHA color was 40. Activated carbon (37.1 g, 3% w/w) was added, and the mixture was heated at 75-80° C. under vacuum (50-70 torr) for 1 h. The mixture was cooled to room temperature and filtered through Celite® to afford 1074 g (86%) of 2-phenylethyl benzoate (99.6% pure by GLC): residual alcohol, <0.05% (GLC); APHA color, 12; acid number, 0.98 mg KOH/g; saponification number, 244 mg KOH/g.

### EXAMPLE 2

### Preparation of 2-Phenylethyl Benzoate (Brønsted Acid Catalyst)

A 2-L, 4-neck, round-bottom flask, fitted with a thermometer, mechanical stirrer, nitrogen inlet tube and Liebig condenser/receiving flask, was charged with 671.7 g (5.50 mol, 1.00 equiv) of benzoic acid, 806.3 g (6.60 mol, 1.20 equiv) of 2-phenylethanol, 2.5 g (0.2% w/w, 0.47 mol %) of methanesulfonic acid (MSA) and 1.25 g (0.1 % w/w) of triisodecylphosphite (TDP). The system was heated gently with slow stirring (<50 rpm) until all the benzoic acid was in solution. The air was removed with three cycles of evacuation/nitrogen fill using a mechanical vacuum pump (50-100 torr). The rate of stirring was increased to ca. 200 rpm, the nitrogen sparge was set at 0.2 scfh, and the reaction mixture was heated to 150° C. After a 1 -h hold, the temperature was increased to 160° C., and the nitrogen sparge was increased to 0.5 scfh. After a 1-h hold, the temperature was increased to 170° C. and held for 2 h. The reaction mixture was cooled to room temperature and sampled for analysis. The acid number was 5.4 mg KOH/g (98.1 % conversion of benzoic acid, corrected for MSA), the APHA color was 49, and the excess 2-phenylethanol was 8.6% by GC. The reaction mixture was heated to 50° C., and 125 g of 10% w/w aqueous sodium carbonate was added. The batch was held at 50° C. and stirred for 15 min. The stirring was stopped and the batch was allowed to settle for 30 min. The aqueous (bottom) layer was removed from the flask with a pipette, and 37.3 g (0.3% w/w) of activated carbon was added. The excess 2-phenylethanol was removed by vacuum distillation at 180-185° C. (20 torr) for 1 h with a nitrogen sweep of 0.5 scfh. The reaction mixture was cooled to room temperature and filtered through Celite® to afford 1030 g (83%) of 2-phenylethyl benzoate (98.7% pure by GLC): residual alcohol, 0.66% (GLC); APHA color, 89; acid number, 0.11 mg KOH/g; saponification number, 241 mg KOH/g.

### EXAMPLE 3

### Preparation of 2-Phenylethyl Benzoate from Benzoyl Chloride

A 2-L, 4-neck, round-bottom flask, fitted with a thermometer, mechanical stirrer, nitrogen inlet tube and Liebig condenser/receiving flask, was charged with 244.3 g (2.00 mol, 1.00 equiv) of 2-phenylethanol, 232.7 g (2.30 mol, 1.15 equiv) of triethylamine, and 376 g of toluene. The rate of stirring set at ca. 200 rpm, the nitrogen sparge was set at 0.1 scfh, and 286.8 g (2.04 mol, 1.02 equiv) of benzoyl chloride was added over a period of 1.5 h, while maintaining the temperature at 10-15° C. The ice bath was removed after an additional 0.5 h at ca. 10° C. and the reaction mixture was allowed to warm to room temperature (23° C.). After 18 h at room temperature, the conversion was 99%, and the 500 g of water was added. After stirring for 30 min at 50° C., the phases were allowed to separate for 15 min, and the aqueous layer (bottom, pH 9) was removed with a pipette. The organic layer was washed with an additional 500 g of water, and the toluene was stripped at 100-105° C. (100 torr). The residue was distilled at 170-172° C. (5 torr) to afford 410 g (91%) of 2-phenylethyl benzoate (99.2% pure by GLC): residual alcohol, 0.08% (GLC); APHA color, 66; acid number, 0.57 mg KOH/g; saponification number, 247 mg KOH/g; refractive index, 1.5576; specific gravity, 1.096.

The process can be run without toluene or similar solvent; however, the reaction mixture tends to become thick and difficult to stir, owing to the precipitation of amine hydrochloride. The solvent also aids phase separation during the aqueous washes.

### EXAMPLE 4

### Preparation of 2-Phenylethyl Benzoate from Benzoic Anhydride

A 1-L, 4-neck, round-bottom flask, fitted with a thermometer, mechanical stirrer, nitrogen inlet tube and Liebig condenser/receiving flask, was charged with 294.1 g (1.30 mol, 1.00 equiv) of benzoic anhydride, 349.4 g (2.86 mol, 2.20 equiv) of 2-phenylethanol, and 1.18 g of Fascat 2001®. The system was heated gently with slow stirring (<50 rpm) until the benzoic anhydride dissolved. The air was removed with three cycles of evacuation/nitrogen fill using a mechanical vacuum pump (50-100 torr). The rate of stirring was increased to ca. 200 rpm, the nitrogen sparge was set at 0.1 scfh, and the reaction mixture was heated to 210° C. After a 1-h hold at 210° C., the amount of distillate was 24.4 g, from which 9.5 g of alcohol was separated and returned to the reaction mixture. The temperature was increased to 220° C. for 1 h, during which time an additional 10.8 g of distillate was collected. The alcohol (3.7 g) was separated and returned to the reaction mixture. The temperature was increased to 230° C., and after a 1-h hold, an additional 1.8 g of distillate had been collected; the alcohol (0.5 g) was not returned. The acid number was 2.15 mg KOH/g. The excess alcohol was stripped and the product was treated with activated carbon as usual to give 470 g (80%) of 2-phenylethyl benzoate (99.4% pure by GLC). The residual alcohol was 0.08% by GLC and the APHA color was 426. The product was distilled as in Example 3 to obtain 430 g (73%) of 2-phenylethyl benzoate (99.7% by GLC): residual alcohol, 0.03% (GLC); APHA color, 10; acid number, 0.21 mg KOH/g; saponification number, 244 mg KOH/g; refractive index, 1.5575; specific gravity, 1.095.

### EXAMPLE 5

### Preparation of 2-Phenylethyl Benzoate (Ester Exchange)

A 1-L, 4-neck, round-bottom flask, fitted with a thermometer, mechanical stirrer, nitrogen inlet tube and reflux condenser, was charged with 492.6 g (3.00 mol, 1.50 equiv) of propyl benzoate, 244.3 g (2.00 mol, 1.00 equiv) of 2-phenylethanol, 2.3 g of Fascat 2001® (tin oxalate) and 2.3 g of Fascat® 4201 (dibutyltin oxide). The rate of stirring was set at ca. 200 rpm, the nitrogen sparge was set at 0.2 scfh, and the reaction mixture was heated at 150-160° C. for 1 h, whereupon reflux commenced. The refux condenser was replaced with a Liebig condenser/receiving flask, and distillate was removed for 30 min at 160° C. with a nitrogen flow of 0.3 scfh. The temperature was increased to 170° C., the nitrogen flow was increased to 0.4 scfh, and distillation (90-95° C. vapor temperature) was continued for 30 min. The temperature was increased by 10° C. and the nitrogen sparge by 0.1 scfh every 30 min until the temperature was 230° C., and a total of 119 g of distillate had been collected (theor. 120 g). The excess propyl benzoate was stripped, and the product was distilled as in Example 3 to obtain 390 g (86%) of 2-phenylethyl benzoate (99.6% pure by GLC): residual 2-phenylethanol, <0.01 % (GLC); residual propyl benzoate, 0.1% (GLC); APHA color, 24; acid number, 0.20 mg KOH/g; saponification number, 245 mg KOH/g; refractive index, 1.5574; specific gravity, 1.095.

### EXAMPLE 6

### Preparation of 1-Phenylethyl Benzoate

The product (98.9% pure by GLC) was prepared from 1-phenylethanol and benzoyl chloride by the method of Example 3: acid number, 1.44 mg KOH/g; saponification number, 248 mg KOH/g; refractive index, 1.5555; specific gravity, 1.092.

### EXAMPLE 7

### Preparation of Benzyl Benzoate

The product (99.3% pure by GLC) was prepared from benzyl alcohol and benzoic acid by the method of Example 1: acid number, 0.37 mg KOH/g; saponification number, 261 mg KOH/g; refractive index, 1.5661; specific gravity, 1.117.

### EXAMPLE 8

### Preparation of p-Methylbenzyl Benzoate

The product (99.0% pure by GLC) was prepared from p-methylbenzyl alcohol and benzoic acid by the method of Example 1: acid number, 0.10 mg KOH/g; saponification number, 239 mg KOH/g; refractive index, 1.5597; specific gravity, 1.003.

### EXAMPLE 9

### Preparation of 3-Phenylpropyl Benzoate

The product (99.7% pure by GLC) was prepared from 3-phenylpropanol and benzoic acid by the method of Example 1: acid number, 0.19 mg KOH/g; saponification number, 232 mg KOH/g; refractive index, 1.5515; specific gravity, 1.078.

### EXAMPLE 10

### Preparation of 2-Phenoxyethyl Benzoate

The product (99.4% pure by GLC) was prepared from 2-phenoxyethanol and benzoic acid by the method of Example 1: acid number, 0.25 mg KOH/g; saponification number, 229 mg KOH/g; refractive index, 1.5608; specific gravity, 1.157.

### EXAMPLE 11

### Preparation of 4-Phenylbutyl Benzoate

The product (99.7% pure by GLC) was prepared from 4-phenylbutanol and benzoic acid by the method of Example 1: acid number, 0.05 mg KOH/g; saponification number, 220 mg KOH/g; refractive index, 1.5467; specific gravity, 1.063.

### EXAMPLE 12

### Preparation of 1-Phenylpropyl Benzoate

The product (98.4% pure by GLC) was prepared from 1-phenylpropanol and benzoyl chloride by the method of Example 3: acid number, 0.96 mg KOH/g; saponification number, 233 mg KOH/g; refractive index, 1.5494; specific gravity, 1.074.

### EXAMPLE 13

### Preparation of 2-(N-benzyl-N-methylamino)ethyl Benzoate

The product (98.1% pure by GLC) was prepared from 2-(N-benzyl-N-methylamino)ethanol and propyl benzoate by the method of Example 5: acid number, 0.65 mg KOH/g; saponification number, 208 mg KOH/g; refractive index, 1.5483; specific gravity, 1.074.

### EXAMPLE 14

### Preparation of Propylene Glycol Dibenzoate

The product (98.9% pure by GLC) was prepared from 1,2-propanediol (propylene glycol) and benzoic acid by the method of Example 1: acid number, 3.31 mg KOH/g; saponification number, 388 mg KOH/g; refractive index, 1.5433; specific gravity, 1.148.

### EXAMPLE 15

### Preparation of 2-Phenylethyl o-Anisate

The product (97.0% pure by GLC) was prepared from 2-phenylethanol and anisic acid by the method of Example 1: acid number, 2.96 mg KOH/g; saponification number, 218 mg KOH/g; refractive index, 1.5646; specific gravity, 1.139.

### EXAMPLE 16

### Preparation of 2-Phenylethyl p-Fluorobenzoate

The product (99.2% pure by GLC) was prepared from 2-phenylethanol and p-fluorobenzoic acid by the method of Example 1: acid number, 0.27 mg KOH/g; saponification number, 227 mg KOH/g; refractive index, 1.5425; specific gravity, 1.158.

### EXAMPLE 17

### Preparation of 2-Phenylethyl o-Toluate

The product (97.2% pure by GLC) was prepared from 2-phenylethanol and o-toluic acid by the method of Example 1: acid number, 0.01 mg KOH/g; saponification number, 225 mg KOH/g; refractive index, 1.5556; specific gravity, 1.082.

### EXAMPLE 18

### Preparation of 1-Phenylethyl o-Toluate

The product (98.0% pure by GLC) was prepared from 1-phenylethanol and o-toluic acid by the method of Example 3: acid number, 0.12 mg KOH/g; saponification number, 231 mg KOH/g; refractive index, 1.5543; specific gravity, 1.079.

### EXAMPLE 19

### Preparation of 2-Phenylethyl p-Toluate

The product (96.1 % pure by GLC) was prepared from 2-phenylethanol and p-toluic acid by the method of Example 1: acid number, 0.15 mg KOH/g; saponification number, 228 mg KOH/g; refractive index, 1.5547; specific gravity, 1.074.

### EXAMPLE 20

### Preparation of 1-Phenylethyl p-Toluate

The product (98.5% pure by GLC) was prepared from 1-phenylethanol and p-toluic acid by the method of Example 3: acid number, 1.50 mg KOH/g; saponification number, 234 mg KOH/g; refractive index, 1.5539; specific gravity, 1.069.

### EXAMPLE 21

### Preparation of 2-Phenylethyl Phenylacetate

The product (98.6% pure by GLC) was prepared from 2-phenylethanol and phenylacetic acid by the method of Example 1: acid number, 0.16 mg KOH/g; saponification number, 231 mg KOH/g; refractive index, 1.5472; specific gravity, 1.081.

### EXAMPLE 22

### Preparation of 1-Phenylethyl Phenylacetate

The product (98.6% pure by GLC) was prepared from 1-phenylethanol and phenylacetyl chloride by the method of Example 3: acid number, 1.39 mg KOH/g; saponification number, 228 mg KOH/g; refractive index, 1.5434; specific gravity, 1.073.

### EXAMPLE 23

### Preparation of 2-Methyl-1-phenyl-2-propyl Phenylacetate

The product (95.3% pure by GLC, 2:1 mixture of isomers) was prepared from 2-methyl-1-phenyl-2-propanol and phenylacetic acid by the method of Example 3: acid number, 9.22 mg KOH/g; saponification number, 173 mg KOH/g; refractive index, 1.5438; specific gravity, 1.053.

### EXAMPLE 24

### Preparation of 2-Phenylethyl 2-Phenylbutyrate

The product (99.7% pure by GLC) was prepared from 2-phenylethanol and 2-phenylbutyric acid by the method of Example 1: acid number, 0.26 mg KOH/g; saponification number, 207 mg KOH/g; refractive index, 1.5351; specific gravity, 1.047.

### EXAMPLE 25

### Preparation of Benzyl α,α,α-Trifluoro-m-tolylacetate

The product (99.4% pure by GLC) was prepared from benzyl alcohol and α,α,α-trifluoro-m-toluic acid by the method of Example 1: acid number, 0.07 mg KOH/g; saponification number, 189 mg KOH/g; refractive index, 1.5054; specific gravity, 1.233.

### EXAMPLE 26

### Preparation of 3-Phenylpropyl Hydrocinnamate

The product (99.6% pure by GLC) was prepared from 3-phenylpropanol and hydrocinnamic acid by the method of Example 1: acid number, 0.12 mg KOH/g; saponification number, 206 mg KOH/g; refractive index, 1.5379; specific gravity, 1.052.

### EXAMPLE 27

### Preparation of 3-Phenylpropyl Phenoxyacetate

The product (99.5% pure by GLC) was prepared from 3-phenylpropanol and phenoxyacetic acid by the method of Example 1: acid number, 0.05 mg KOH/g; saponification number, 206 mg KOH/g; refractive index, 1.5454; specific gravity, 1.111.

### EXAMPLE 28

### Preparation of Dibenzyl Malonate

The product (97.9% pure by GLC) was prepared from benzyl alcohol and dimethyl malonate by the method of Example 5: acid number, 0.43 mg KOH/g; saponification number, 387 mg KOH/g; refractive index, 1.5415; specific gravity, 1.161.

### EXAMPLE 29

### Solubility of Solid Organic Sunscreens in Various Solvents

Predetermined solutions (w/w) were prepared at 40-50° C. using a given solvent-sunscreen combination. The solutions were allowed to stand for 1 week at 25° C. in a constant temperature chamber. A small seed crystal was initially added at 25° C. to hasten equilibration. Solubility was measured by GLC using standard solutions to calibrate the instrument. As shown below in Table 1, the solubilizer of the invention is effective in solubilizing at least 10%, preferably 20%, most preferably 30% or more (w/w) of at least one of the sunscreens.

TABLE 1
Solubility data for sunscreen compounds.

| | Sunscreen | | |
|---|---|---|---|
| Solvent | Avobenzone | Oxybenzone | MBC |
| 2-phenylethyl benzoate | 26 | 36 | 39 |
| 1-phenylethyl benzoate | 26 | 35 | 40 |
| benzyl benzoate | 28 | 38 | 40 |
| p-methylbenzyl benzoate | 30 | 36 | 39 |
| 3-phenylpropyl benzoate | 31 | 35 | 38 |
| 2-phenoxyethyl benzoate | a | 35 | a |
| 4-phenylbutyl benzoate | 27 | 32 | 37 |
| 1-phenylpropyl benzoate | 23 | 32 | 39 |
| 2-(N-benzyl-N-methylamino)ethyl | 25 | 31 | 35 |
| benzoate | | | |
| propylene glycol dibenzoate | 21 | 32 | 33 |
| 2-phenylethyl o-anisate | 26 | 33 | 32 |
| 2-phenylethyl p-fluorobenzoate | 23 | 33 | 37 |
| 2-phenylethyl o-toluate | 24 | 33 | 40 |
| 1-phenylethyl o-toluate | 25 | 32 | 39 |
| 2-phenylethyl p-toluate | 26 | 33 | 39 |
| 1-phenylethyl p-toluate | 34 | 34 | 39 |
| 2-phenylethyl phenylacetate | 17 | 35 | 33 |
| 1-phenylethyl phenylacetate | 17 | 34 | 35 |
| 2-methyl-1-phenyl-2-propyl | 23 | 34 | 37 |
| phenylacetate | | | |
| 2-phenylethyl 2-phenylbutyrate | 20 | 30 | 34 |
| benzyl α,α,α-trifluoro-m-tolylacetate | 15 | 28 | 34 |
| 3-phenylpropyl hydrocinnamate | 20 | 33 | 33 |
| 3-phenylpropyl phenoxyacetate | 17 | 33 | 31 |
| dibenzyl malonate | 14 | 31 | 25 |
| C₁₂₋₁₅ alkyl benzoate | 16 | 16 | 29 |
| (Finsolv TN ® control) | | | |

^{a}Entire mixture solidified.

### EXAMPLE 30

Anhydrous Oil Sunscreen Composition

| Phase | Ingredient | % w/w |
|---|---|---|
| A | Ceraphyl 368 (Ethylhexyl Palmitate) | 5.00 |
| | Escalol 567 (Oxybenzone) | 3.00 |
| | Escalol 517 (Avobenzone) | 3.00 |
| | Ceraphyl 41 (C₁₂₋₁₅ Alkyl Lactate) | 20.00 |
| | X-Tend 226 | 20.00 |
| | Escalol 597 (Octocrylene) | 1.50 |
| | Escalol 587 (Octisalate) | 5.00 |
| | Ceraphyl 55 | 5.00 |
| | Escalol 557 (Octinoxate) | 7.50 |
| | Ganex V-216 (PVP-Hexadecene Copolymer) | 3.00 |
| | Vitamin E Acetate (Tocopheryl Acetate) | 0.10 |
| B | Si Tec DM 1 Plus (Dimethicone) | 5.00 |
| | Si Tec PTM 200 | 3.00 |
| | Si Tec CM 040 | 17.70 |
| C | Liquapar Optima (Phenoxyethanol and Methylparaben | 1.00 |
| | And Isopropylparaben and isobutylparaben and | |
| | Butylparaben) | |
| | Suncare Fragrance RR 82895 | 0.20 |
| | | 100.00 |

Procedure: The phase A ingredients were combined and mixed with moderate stirring at 70° C. until homogeneous. The batch was cooled to 50° C., and the phase B ingredients were added, mixing after each addition until clear. At 40° C., the phase C ingredients were added, and the batch was mixed until clear.

SPF=22.8, which is significantly higher than the value for the control in Example 31.

### EXAMPLE 31

Anhydrous Oil Sunscreen Composition (Control)

| Phase | Ingredient | % w/w |
|---|---|---|
| A | Ceraphyl 368 (Ethylhexyl Palmitate) | 5.00 |
| | Escalol 567 (Oxybenzone) | 3.00 |
| | Ceraphyl 55 (Tridecyl Neopentanoate) | 25.00 |
| | Escalol 517 (Avobenzone) | 3.00 |
| | Ceraphyl 41 (C₁₂₋₁₅ Alkyl Lactate) | 20.00 |
| | Escalol 587 (Octisalate) | 5.00 |
| | Escalol 597 (Octocrylene) | 1.50 |
| | Escalol 557 (Octinoxate) | 7.50 |
| | Ganex V-216 (PVP-Hexadecene Copolymer) | 3.00 |
| | Vitamin E Acetate (Tocopheryl Acetate) | 0.10 |
| B | Si Tec DM 1 Plus (Dimethicone) | 5.00 |
| | Si Tec PTM 200 (Phenyl Trimethicone) | 3.00 |
| | Si Tec CM 040 (Cyclopenthasiloxane) | 17.70 |
| C | Liquapar Optima (Phenoxyethanol and Methylparaben | 1.00 |
| | and Isopropylparaben and Isobutylparaben and | |
| | Butylparaben) | |
| | Suncare Fragrance RR 82895 Ungerer | 0.20 |
| | | 100.00 |

Procedure: The phase A ingredients were combined and mixed with moderate stirring at 70° C. until homogeneous. The batch was cooled to 50° C., and the phase B ingredients were added, mixing after each addition until clear. At 40° C., the phase C ingredients were added, and the batch was mixed until clear.

SPF=12.0 was measured.

### EXAMPLE 32

### Enhancement of UVA Absorption

A 10-mg portion of sunscreen was dissolved in 1 L of solvent, and the UV spectrum of the solution was measured using a Cary 1 E UV-Visible spectrophotometer. The results in Table 2 demonstrate that greater UVA protection is afforded for the active sunscreen using 2-phenylethyl benzoate instead of C₁₂₋₁₅ benzoate in the composition.

TABLE 2
UVA Absorption Data

λₘₐₓ, nm

| Solvent | E-517 | E-567 |
|---|---|---|
| Ethanol | 358 | 325 |
| C₁₂₋₁₅ benzoate (Finsolv ® TN) | 358 | 328 |
| 2-phenylethyl benzoate | 362 | 329 |

### EXAMPLE 33

### Broad Spectrum UVA/UVB Sunscreen Formulations

These ‘anti-aging’ formulations (Table 3) were examined for critical wavelength, a measure of UVA protection, using an Optometrics SPF 290 analyzer after five freeze-thaw cycles and then after 1 month of storage at 45° C. The higher the critical wavelength, the greater the UVA protection. As can be seen for both the freeze-thaw and 1-month storage conditions (Table 4), the formulation containing X-Tend® 226 (2-phenylethyl benzoate) was superior to the other formulations containing Finsolv® TN, Eldew® SL-205, Finsolv® TPP, and Elefac® I-305.

TABLE 3
Anti-aging cream formulations.

| | Formulation | | | | |
|---|---|---|---|---|---|
| | 1 | 2 | 3 | 4 | 5 |
| Ingredient | % w/w | % w/w | % w/w | % w/w | % w/w |
| Phase A | | | | | |
| Deionized water | 57.35 | 57.35 | 57.35 | 57.35 | 57.35 |
| Stabileze ® QM | 0.50 | 0.50 | 0.50 | 0.50 | 0.50 |
| Butylene glycol | 3.00 | 3.00 | 3.00 | 3.00 | 3.00 |
| Disodium EDTA | 0.10 | 0.10 | 0.10 | 0.10 | 0.10 |
| Phase B | | | | | |
| Cerasynt ® 840 | 1.50 | 1.50 | 1.50 | 1.50 | 1.50 |
| Cerasynt ® 945 | 2.00 | 2.00 | 2.00 | 2.00 | 2.00 |
| Escalol ® 557 | 7.50 | 7.50 | 7.50 | 7.50 | 7.50 |
| Escalol ® 517 | 3.00 | 3.00 | 3.00 | 3.00 | 3.00 |
| Escalol ® 587 | 5.00 | 5.00 | 5.00 | 5.00 | 5.00 |
| Escalol ® 567 | 2.00 | 2.00 | 2.00 | 2.00 | 2.00 |
| X-Tend ® 226 | 10.00 | 0.00 | 0.00 | 0.00 | 0.00 |
| Finsolv ® TN | 0.00 | 10.00 | 0.00 | 0.00 | 0.00 |
| Eldew ® SL-205 | 0.00 | 0.00 | 10.00 | 0.00 | 0.00 |
| Finsolv ® TPP | 0.00 | 0.00 | 0.00 | 10.00 | 0.00 |
| Elefac ® I-305 | 0.00 | 0.00 | 0.00 | 0.00 | 10.00 |
| Phase C | | | | | |
| Sodium hydroxide, 10% | 1.30 | 1.30 | 1.30 | 1.30 | 1.30 |
| w/w | | | | | |
| Deionized water | 5.00 | 5.00 | 5.00 | 5.00 | 5.00 |
| Phase D | | | | | |
| Liquapar ® Optima | 1.25 | 1.25 | 1.25 | 1.25 | 1.25 |
| Liquapar ® Oil | 0.40 | 0.40 | 0.40 | 0.40 | 0.40 |
| Phase E | | | | | |
| Glycacil ®-L | 0.100 | 0.100 | 0.100 | 0.100 | 0.100 |
| | 100.00 | 100.00 | 100.00 | 100.00 | 100.00 |

Typical Preparation: For Phase A, a beaker was charged with water, butylene glycol and disodium EDTA. Mixing was begun, and Stabileze® QM was slowly sifted into it. The batch was heated to 80° C. with mixing and held for 45 min. In a separate beaker, the ingredients for Phase B were combined, mixed and heated to 75° C. Phase C was slowly added to Phase A, and the batch was mixed until clarity was obtained, and then Phase B was add. The batch was cooled to 45° C. with mixing, and Phase D was added. After mixing thoroughly, Phase E was added and the batch was again mixed thoroughly. After qs for water loss, it was packaged.

TABLE 4
Critical wavelength data.

Critical
wavelength (nm)

| Formulation | freeze-thaw | storage |
|---|---|---|
| 1 | 376.1 | 375.1 |
| 2 | 374.7 | 373.3 |
| 3 | 374.3 | 374.1 |
| 4 | 373.9 | 373.8 |
| 5 | 373.7 | 372.5 |

### EXAMPLE 34

### Solubility of Triclosan

5-Chloro-2-(2,4-dichlorophenoxy)phenol (Triclosan) has bacteriostatic properties and is used as a disinfectant and preservative in cosmetic and detergent preparations. It is soluble up to 69% w/w in 2-phenylethyl benzoate, as determined by GLC.

An 80% w/w solution prepared from 8.002 g of Triclosan and 2.009 g of 2-phenylethyl benzoate precipitated a significant amount of solid at 25° C. A 23.3-mg sample of the supernatant was dissolved in 1.00 mL of chloroform and 1.00 μL was injected via automatic injector into a GLC instrument. The areas of the 2-phenylethyl benzoate and Triclosan peaks were 9381 and 12953, respectively. The mixture was heated at 70° C. until it was homogeneous, and an 18.2-mg sample was dissolved and injected in the same manner. The 2-phenylethyl benzoate peak had an area of 4456 units, which represented 3.6 μg, and the Triclosan peak had an area of 11240 units, which represented 14.6 μg. (Note that the amount injected was 3.6 μg+14.6 μg=18.2 μg.) Therefore, under our GLC conditions the response factors were 1240 units/μg and 770 units/μg, respectively. Then, the respective amounts of each component in the supernatant were 9381/1240=7.6 μg and 12953/770=16.8 μg, which corresponds to 69% w/w Triclosan.

While the invention has been described with particular reference to certain embodiments thereof, it will be understood that changes and modifications may be made which are within the skill of the art. Accordingly, it is intended to be bound only by the following claims.

1. A composition of an active or functional organic compound solubilized in a diaryl organic compound containing a polar or polarizable functional group therein.

2. A composition according to claim 1 wherein said polar group is an ester.

3. A composition according to claim 1 wherein said diaryl organic compound has the general formula: 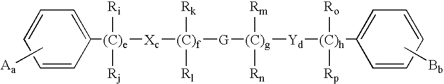
where
G=polar or polarizable functional group (e.g., ester, amide, carbonate, carbamate, urea, carbinyl, oxa, oxo, alkylidene, silyl, sulfonyl, sulfoxyl, phosphonyl, phosphinyl, or thio derivatives thereof).
X_{c}, Y_{d}=G or heteroatom and any attached groups (e.g., O, S, or NR_{q}).
Aₐ, B_{b}=H, F, alkyl or fluoralkyl groups, CN, CO₂Rᵣ, or heterogroups (e.g., OH, ORₛ, O₂CRₜ, NRᵤRᵥ, NO₂, F, Cl, SiR_{w}RₓR_{y}, SO₃R_{z}).
Rᵢ—R_{z}=H, F, alkyl or fluoralkyl groups (e.g., methyl, ethyl, n-propyl, i-propyl, n-butyl, s-butyl, i-butyl, t-butyl, or their fluoro analogs) or alkoxy groups OR′ (R′═Rᵢ—R_{z}).
a, b=1-5
c, d=0-2
e-z=04.

4. A composition according to claim 1 in which said diaryl organic compound is an ester of an aryl carboxylic acid and an aryl alcohol.

5. A composition according to claim 3 in which said diaryl organic compound is an ester having the formula: 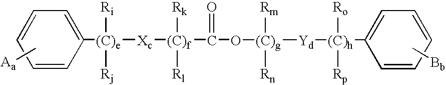
where A, B, X, Y and R are as defined above.

6. A composition according to claim 5 in which said diaryl organic compound has the formula: 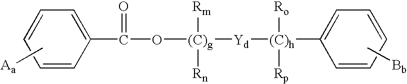
where A, B, Y and R are as defined above.

7. A composition according to claim 6 in which said diaryl organic compound has the formula: 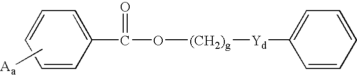
where A and Y are as defined above, and g=1-3.

8. A composition according to claim 5 in which said diaryl organic compound has one of the following named formulas:

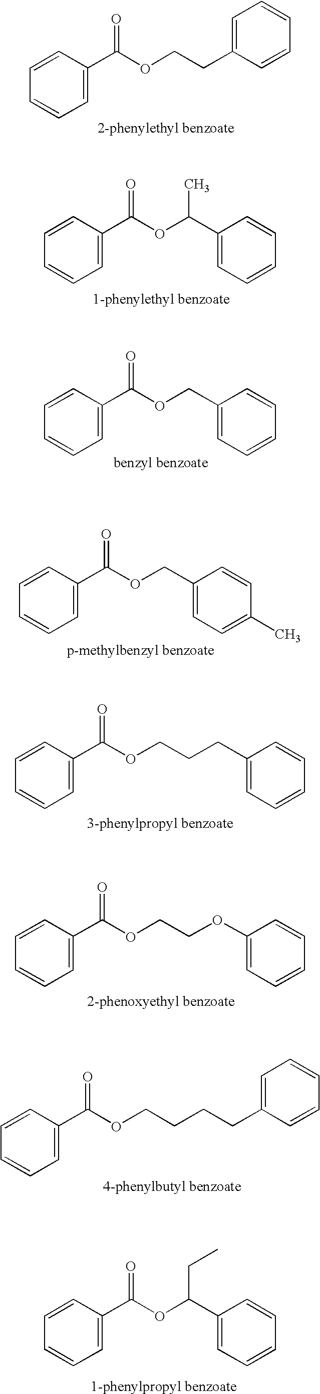

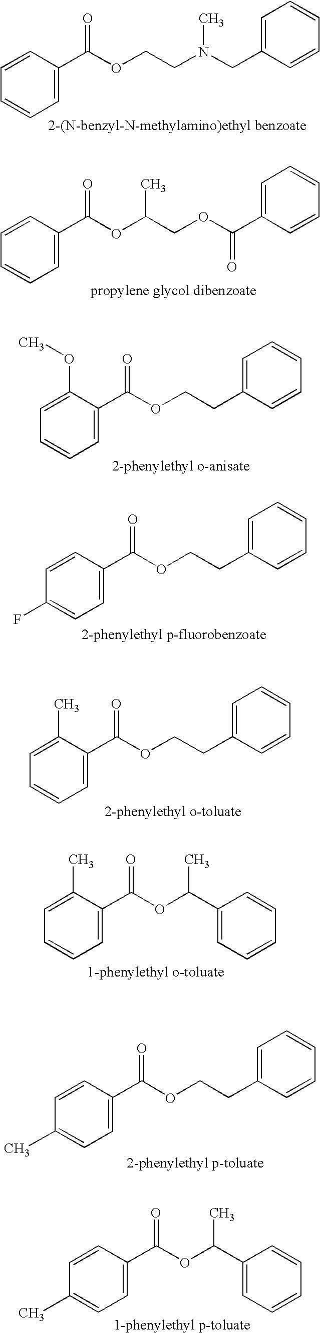

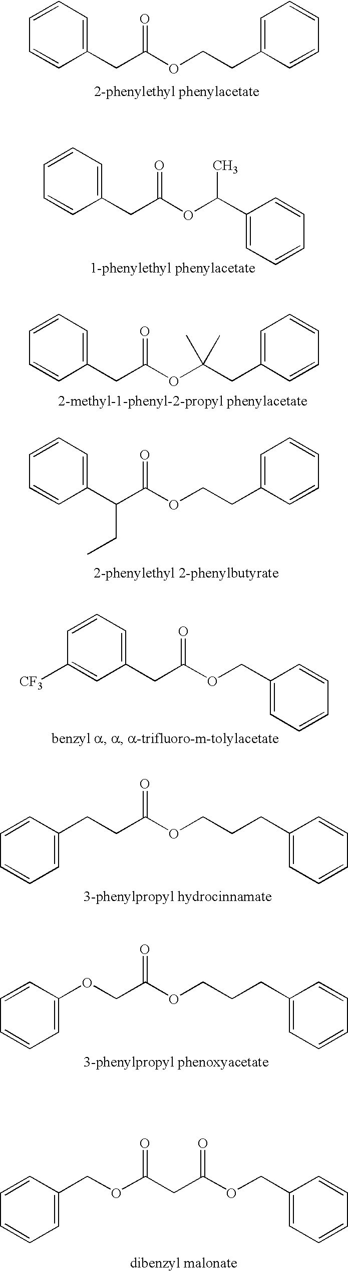

9. A composition according to claim 1 in which said active compound is a solid organic compound.

10. A composition according to claim 1 wherein said active or functional compound is a personal care, cosmetic, pharmaceutical, agricultural or industrial compound.

11. A composition according to claim 10 which is a sunscreen composition.

12. A sunscreen composition according to claim 11 containing UVA and/or UVB chemical compounds, which in said sunscreen composition show increased SPF and/or UVA/UVB absorbance ratio and/or critical wavelength.

13. A sunscreen composition according to claim 11 in which said active compound is solubilized in an amount of at least 20% w/w.

14. A sunscreen composition according to claim 11 in which said active is avobenzone, benzophenone-3, or 4-methylbenzylidene camphor, or mixtures thereof.

15. A sunscreen composition according to claim 13 in which said active is avobenzone, benzophenone-3, or 4-methylbenzylidene camphor, or mixtures thereof.

16. A sunscreen composition according to claim 11 in which said active is selected from the group consisting of Avobenzone, Benzophenone-3, 4-Methylbenzylidene camphor, p-Aminobenzoic acid (PABA), Camphor benzalkonium methosulfate, Homosalate, Phenylbenzimidazole sulfonic acid, Terephthalidene dicamphor sulfonic acid, Benzylidene camphor sulfonic acid, Octocrylene, Polyacrylamidomethyl benzylidene camphor, Ethylhexyl methoxycinnamate, PEG-25 PABA, Isoamyl p-methoxycinnamate, Ethylhexyl triazone, Drometrizole trisiloxane, Diethylhexyl butamido triazone, 3-Benzylidene camphor, Ethylhexyl salicylate, Ethylhexyl dimethyl PABA, Benzophenone-4, Benzophenone-5, Methylene bis-benztriazolyl tetramethylbutylphenol, Disodium phenyl dibenzimidazole tetrasulfonate, Bis-ethylhexyloxyphenol methoxyphenol triazine, Polysilicone-15, and mixtures thereof.

17. A sunscreen composition according to claim 13 in which said active is selected from the group consisting of Avobenzone, Benzophenone-3, 4-Methylbenzylidene camphor, p-Aminobenzoic acid (PABA), Camphor benzalkonium methosulfate, Homosalate, Phenylbenzimidazole sulfonic acid, Terephthalidene dicamphor sulfonic acid, Benzylidene camphor sulfonic acid, Octocrylene, Polyacrylamidomethyl benzylidene camphor, Ethylhexyl methoxycinnamate, PEG-25 PABA, Isoamyl p-methoxycinnamate, Ethylhexyl triazone, Drometrizole trisiloxane, Diethylhexyl butamido triazone, 3-Benzylidene camphor, Ethylhexyl salicylate, Ethylhexyl dimethyl PABA, Benzophenone-4, Benzophenone-5, Methylene bis-benztriazolyl tetramethylbutylphenol, Disodium phenyl dibenzimidazole tetrasulfonate, Bis-ethylhexyloxyphenol methoxyphenol triazine, Polysilicone-1 5, and mixtures thereof.

18. A composition according to claim 1 wherein said active or functional organic compound is selected from the group consisting of Furosemide, Lovastatin, Clarithromycin, Diclofenac, Famotidine, Carbamaxepine, Dipyridamole, Chlorthiazide, Spironolactone, Dilantin, Imipranine, Melfloquine, Cyclosporine, Glyburide, Nimodipine, and mixtures thereof.

19. A composition according to claim 1 wherein said active or functional organic compound is selected from the group consisting of cosmetic, pharmaceutical, agricultural, and industrial compounds.

20. A composition according to claim 1 comprising at least two active or functional organic compounds selected from the group consisting of UV-filter, cosmetic, and pharmaceutical compounds.

21. A composition according to claim 1 comprising a UV-filter compound or a pharmaceutical compound.

22. A composition according to claim 21 comprising a UV-filter compound selected from the group consisting of Avobenzone, Benzophenone-3, 4-Methylbenzylidene camphor, p-Aminobenzoic acid (PABA), Camphor benzalkonium methosulfate, Homosalate, Phenylbenzimidazole sulfonic acid, Terephthalidene dicamphor sulfonic acid, Benzylidene camphor sulfonic acid, Octocrylene, Polyacrylamidomethyl benzylidene camphor, Ethylhexyl methoxycinnamate, PEG-25 PABA, Isoamyl p-methoxycinnamate, Ethylhexyl triazone, Drometrizole trisiloxane, Diethylhexyl butamido triazone, 3-Benzylidene camphor, Ethylhexyl salicylate, Ethylhexyl dimethyl PABA, Benzophenone-4, Benzophenone-5, Methylene bis-benztriazolyl tetramethylbutylphenol, Disodium phenyl dibenzimidazole tetrasulfonate, Bis-ethylhexyloxyphenol methoxyphenol triazine, Polysilicone-15, and mixtures thereof, and a pharmaceutical compound selected from the group consisting of Furosemide, Lovastatin, Clarithromycin, Diclofenac, Famotidine, Carbamaxepine, Dipyridamole, Chlorthiazide, Spironolactone, Dilantin, Imipranine, Melfloquine, Cyclosporine, Glyburide, Nimodipine, and mixtures thereof.
